(19) United States
(12) Patent Application Publication
(10) Pub. No.: US 2004/0022674 A1
(43) Pub. Date: Feb. 5, 2004

(54) Sterile containers

(75) Inventors: Jurgen Thurk, St. Gallen (CH); Josef Schneider, St. Gallen (CH)

Correspondence Address: CHARLES N.J. RUGGIERO, ESQ., OHLANDT, GREELEY, RUGGIERO & PERLE, L.L.P., ONE LANDMARK SQUARE, 10th FLOOR, STAMFORD, CT, 06901-2682, US

(73) Assignee: Schott Glas

(21) Appl. No.: 10/386,040

(22) Filed: Mar. 11, 2003

Related U.S. Application Data
(63) Continuation of application No. PCT/EP01/10239, filed on Sep. 6, 2001.

(30) Foreign Application Priority Data
Sep. 12, 2000 (DE) DE 100 45 334.1

(51) Int. Cl. A61L 2/20

(52) U.S. Cl. 422/28; 422/292

(57) ABSTRACT

A sterile packaging is provided. The sterile packaging includes one or several containers packaged in a gas-permeable and microbiologically impervious manner for sterilizing by means of plasma.

The invention relates to sterile containers as well as the use of the sterile containers as a primary packaging in the pharmaceutical industry.

From EP-A-801 952 a method for sterilizing containers by means of low-pressure plasma is known. In the case of containers such as bottles which constrict towards a filling opening, the plasma ignited in the interior of the container can become inhomogeneous in the zone of the filling opening. This may lead under certain circumstances not only to an undesirable heating of the container in the zone of the filling opening, but can also decrease the efficiency of the sterilization process. Moreover, if the plasma burns out of the filling opening this can lead to a damage of the reactor.

From DE-A-196 15 735 an apparatus is known for sterilizing containers by means of low-pressure plasma treatment. In the case of high outputs it is possible that constricting cross sections can become hot. The plasma which is ignited essentially only in the interior of the container burns out of the filling opening and destroys the ambient region.

Several methods are known for sterilizing pharmaceutical packaging material. The well-known method for sterilizing with ethylene oxide (ETO) is toxic, has long process periods and is polluting to the environment. In the other known methods of sterilization with gamma rays there is discoloration of the packaging material made of glass and plastic and an embrittlement of rubber.

From U.S. Pat. No. 5,879,620 a device for sterilization comprising an inner sterilization wrap and a reusable outer sterilization bag is known. Since the sterilization bag is intended to be re-used, after sterilization has been completed the objects are removed from the sterilization bag before e.g. shipping to the customer.

From U.S. Pat. No. 6,021,625 a method of vented foil packaging for medical devices is disclosed. The packaging known from U.S. Pat. No. 6,021,625 is sterilized by using conventional steriliant gases such as ethylene oxide gas.

Object of the invention is to overcome the disadvantages of the prior art and to provide a sterile packaging.

The object of the present invention is achieved by a sterile packaging, with one or several containers being packaged in a gas-permeable and microbiologically impervious manner and for sterilizing by plasma.

In a preferred embodiment of the invention the sterile packaging of one or several containers comprises at least one gas-permeable and microbiologically impervious sealing and at least one gas-permeable and microbiologically impervious wrap-packaging. The containers thus sterilized can be sent in a sealed and packaged fashion to the user. It was noticed that very favorable results are achieved during the sterilization of the containers in a packaging sealed and wrapped in accordance with the invention.

The containers provided for sterilization are situated preferably in a solid or flexible device for receiving the one or several containers. The device is sealed by a gas-permeable and microbiologically impervious lid. The sealed device is wrapped in addition with at least one gas-permeable and microbiologically impervious wrap-packaging.

In the packaging in accordance with the invention the interior surface as well as the outer surfaces even of heat-sensitive containers are sterilized by means of a low-pressure plasma in an evacuable reactor. The reactor contains a vacuum pump for evacuating the reactor, a feed line for the ionizing gas and the plasma-producing electrodes.

In accordance with the invention, a terminal stage of the sterilization is provided, with the containers sealed and packaged being sterilized within the wrap-packaging. The thus wrap-packaged containers are successfully sterilized and can be shipped.

In accordance with the invention, containers are preferably sterilized for the packaging of solid or liquid diagnostic agents and pharmaceuticals for highest demands in the pharmaceutical industry. Such containers are ampules, small bottles, carpules, syringe cylinders and other glass and/or glass and/or plastic parts which contain rubber and/or metal. Also syringes with syringe cylinders, needle, protective needle caps and wrap-packaging are also provided for sterilization in the inventive sterile packaging.

Preferably, the containers are syringes without needles and without protective caps, syringes with needles and without protective caps or syringes with needles and with protective caps or syringes without needles and with protective caps. Especially preferably are syringes with syringe cylinders, needles and finger rests. Most preferably are containers that comprise in addition piston stoppers, finger rests and backstops.

The advantages of the applied process for sterilization of the containers are the short process time. No residual substances in the ppm region remain in the container. The process is performed dry and without odor. No toxic chemicals are used in the process and no disposal problem arises. There is no thermal encumbrance and no embrittlement of the container. The method in accordance with the invention is similarly suitable for containers that comprise glass, plastic, rubber or metal materials and leads to very favorable results.

One or several containers according to the invention can be sealed in a packaging and additionally sterilized in a welded wrap-packaging. In the sealed and welded wrap-packaging very favorable results are achieved during sterilization.

The device for receiving one or several containers is preferably made of polyethylene HDPE and can be sealed in an embodiment of the invention, e.g. by Tyvek®. Tyvek® is produced by Dupont and consists to 100% of polyethylene HDPE.

The welded wrap-packagings comprise a transparent plastic film on the one side and polyethylene HDPE on the other side, which preferably is Tyvek®. The plastic film contains polyethylene, polypropylene or cyclo-olefine polymer such as ethylene norborn co-polymer.

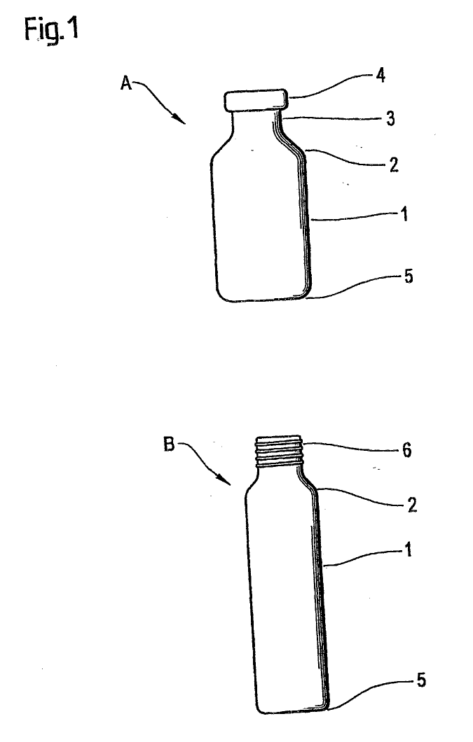

The invention is now explained in closer detail by reference to the enclosed drawings, wherein the drawings consist of FIGS. 1 to 4:

FIG. 1 shows small bottles A and B as an example for the containers received by the inventive sterile packaging;

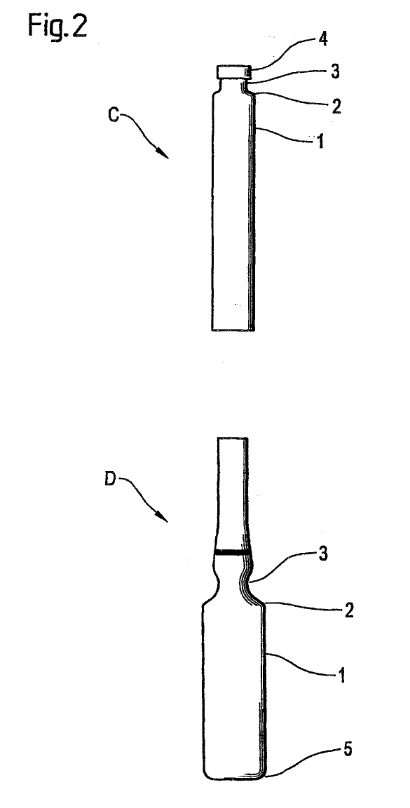

FIG. 2 shows a carpule C and an ampule D as an example for the containers received by the inventive sterile packaging;

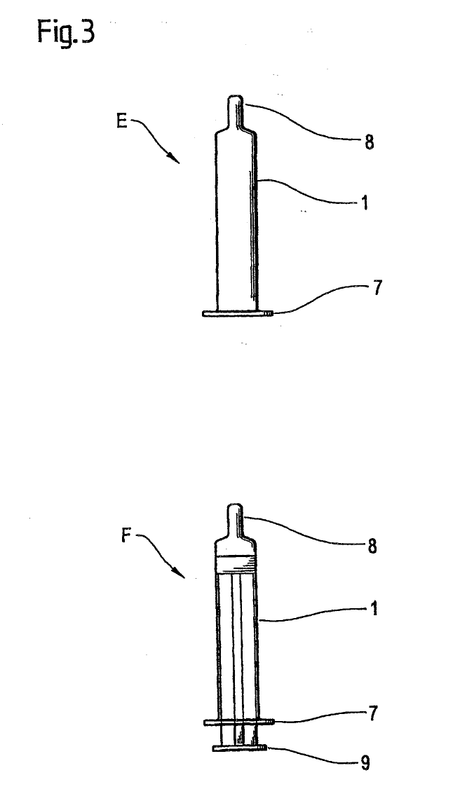

FIG. 3 shows a syringe cylinder E and a syringe cylinder with a piston F as an example for the containers received by the inventive sterile packaging;

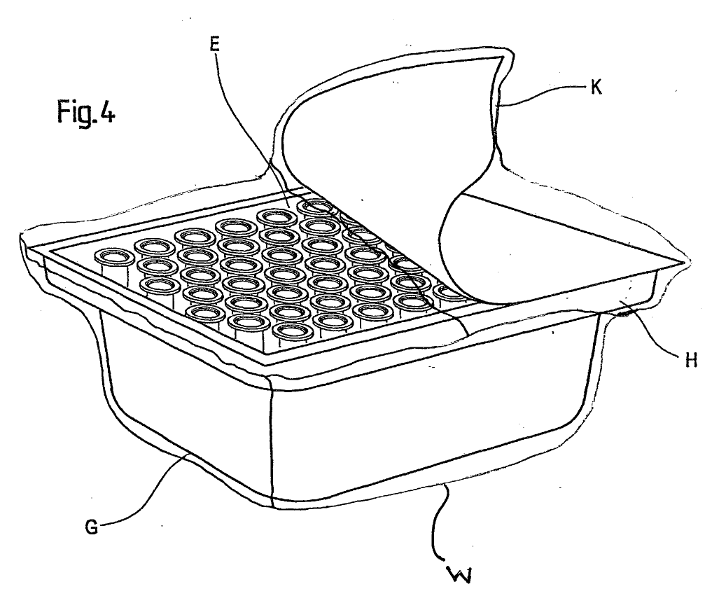

FIG. 4 shows a filled plastic trough G that is to be sealed, comprising a nest H, syringes E and a gas-permeable and microbiologically impervious lid K and a wrap-packaging W.

In FIG. 1, reference A shows a small bottle with the body (1), shoulder (2), throat (3), collar (4) and a floor (5). B shows a screw cap bottle comprising a thread (6) for a screw cap.

FIG. 2 shows with reference C a carpule comprising a body (1), shoulder (2), throat (3), collar (4), bottom-floor. D shows an ampule comprising a body (1), shoulder (2), neck (3) and bottom-floor (5).

FIG. 3 shows a cylindrical body with a syringe comprising a body (1), finger rest (7) and a cone (8). F shows a syringe cylinder with a piston rod (9). The syringe can be equipped with needle and cap. It is also possible to pre-fill the sterilized syringe with a pharmaceutical and to store the same as a pre-filled syringe.

FIG. 1 to FIG. 3 shows examples for the containers, which are received by the inventive sterile packaging. In FIG. 4 an inventive sterile packaging is shown.

FIG. 4 shows a plastic trough G which is filled with syringes E and is gas-permeable and is to be sealed thoroughly in a microbiological respect. A nest H is inserted at first into the plastic trough. Syringes E are inserted into the nest H. The plastic trough G is sealed with a gas-permeable and microbiologically impervious lid K. The sealed plastic trough is thus a gas-permeable and microbiologically impervious packaging container. The sealed plastic trough is additionally packaged with at least one gas-permeable and microbiologically impervious wrap-packaging W is shown.

### EXAMPLES

The sterilization effect in the containers per se and the packaging material was examined.

The table below shows an overview of the results which were achieved during the sterilization tests. The germ reduction figures are stated in magnitudes or log stages. A sign > before the numerical value means that the actually achieved sterilization result is higher than the stated value. In these cases the germ reduction was so high or the survival probability of the germs so low that in the given preliminary microbic contamination no germs survived the sterilization process.

TABLE
Overview of results

| Examination groups | Test objects | Results |
|---|---|---|
| Containers sealed | Outside of container in Tyvek, | >6.0 |
| and packaged | wrap-packaging | |
| | Inside of needle with caps | >5.3 |
| | Outside of needle with caps | >6.3 |

The tests have shown that the necessary sterilization effect is achieved when the containers are sealed and packaged in a sterile packaging according to the invention.

1. A sterile packaging, wherein one or several containers are packaged in a gas-permeable and microbiologically impervious manner for sterilizing by means of plasma.

2. A packaging according to claim 1, wherein the packaging of one or several containers comprises at least one gas-permeable and microbiologically impervious sealing and furthermore at least one gas-permeable and microbiologically impervious wrap-packaging.

3. A packaging according to one of the claims 1 or 2, comprising at least a material such as glass, plastic, rubber or metal.

4. A packaging according to one of the claims 1 to 3, wherein the sealing is made of one or several layers.

5. A packaging according to one of the claims 1 to 4, wherein the container is an ampule, carpule or a syringe.

6. A packaging according to one of the claims 1 to 5, wherein the container is a syringe without needle and without protective cap, with needle and without protective cap or with needle and with protective cap.

7. A packaging according to one of the claims 1 to 6, wherein the container is a syringe and the syringe comprises syringe cylinder, needle and finger rest.

8. A packaging according to one of the claims 1 to 7, wherein the container is a syringe and the syringe additionally comprises a piston stopper, finger rest and backstop.

9. A sterile packaging for one or several containers which is made sterile by plasma sterilization comprising
a device for receiving one or several containers, wherein said device is sealed by a seal in a gas-permeable and microbiologically impervious manner; and
a wrap-packaging wherein said wrap-packaging encloses said device in a gas-permeable and microbiologically impervious manner.

10. A sterile packaging according to claim 9,
wherein said device comprises a plastic trough and a nest for receiving said one or several containers.

11. A sterile packaging according to claim 9,
wherein
said wrap-packaging is a welded packaging and said welded packaging comprises a transparent plastic film on one side and HDPE-polyethylene on the other side.

12. A sterile packaging according to claim 11,
wherein said transparent plastic film comprises polyethylene, polypropylene or a cyclo-olefine polymer.

13. The use of a sterile packaging according to claim 9 as a primary packaging in the pharmaceutical industry.

14. The use of a sterile packaging according to claim 9 as a packaging of solid or liquid diagnostic agents and pharmaceuticals, foodstuffs and cosmetics.
